(19) United States
(12) Patent Application Publication
(10) Pub. No.: US 2022/0402825 A1
(43) Pub. Date: Dec. 22, 2022

(54) METHOD FOR PRODUCING TOUGHENED ZIRCONIA MATERIALS FOR PROSTHESES

(71) Applicant: Xylon Technical Ceramics, Inc., Alfred, NY (US)

(72) Inventors: Nicholas H. Burlingame, Belmont, NY (US); Samuel J. Burlingame, Belmont, NY (US)

(73) Assignee: Xylon Technical Ceramics, Inc., Alfred, NY (US)

(21) Appl. No.: 17/838,591

(22) Filed: Jun. 13, 2022

Related U.S. Application Data
(60) Provisional application No. 63/211,108, filed on Jun. 16, 2021.

(51) Int. Cl. C04B 35/486; A61L 31/02

(52) U.S. Cl. CPC C04B 35/486; A61L 31/026; C04B 2235/3208; C04B 2235/3246

(57) ABSTRACT

A disclosure is provided for methods to prepare high-strength and high-toughness partially stabilized zirconia (PSZ) materials by incorporating a starting ceramic powder in which the stabilizing oxide agent is pre-alloyed with the zirconia material powder. The ceramic powder is pre-stabilized so there is little or no remaining free stabilizing oxide, thereby resulting in an improved material that is more convenient to process using conventional ceramic processing techniques.

This application claims priority under 35 U.S.C. § 119(e) from U.S. Provisional Patent Application No. 63/211,108, for a “METHOD FOR PRODUCING TOUGHENED ZIRCONIA MATERIALS,” filed Jun. 16, 2021 by Nicholas H. Burlingame and Samuel J. Burlingame, which is hereby incorporated by reference in its entirety.

### CROSS-REFERENCE

Cross-reference is made to the following related patents, which are also hereby incorporated by reference in their entirety:

U.S. Pat. No. 4,279,655 for Partially Stabilized Zirconia Ceramics, which discloses the preparation of Mg-PSZ ceramics produced using Mg-based oxide stabilizing agent and ZrO₂ powder which is not-pre-alloyed;

U.S. Pat. No. 9,162,008 for Ceramic Manufactures, which discloses the production of Mg-PSZ orthopedic implants produced using MgO+ZrO₂ powder which is not pre-alloyed; and

U.S. Pat. No. 8,940,256 for a Method for Recycling of Rare Earth and Zirconium Oxide Materials, disclosing the use of ammonium sulfate to digest rare earth oxide and zirconium oxide to form a soluble salt.

The various embodiments disclosed are directed to the preparation of high-strength and high-toughness partially stabilized zirconia (PSZ) materials by incorporating a starting ceramic powder in which the stabilizing oxide agent is pre-alloyed with the zirconia material powder. The ceramic powder is pre-stabilized and there is no substantial amount of free stabilizing oxide, resulting in an improved material which is more convenient to process using conventional ceramic processing techniques.

### BACKGROUND AND SUMMARY

Partially-stabilized zirconia materials are used for applications that require high-strength and high-toughness materials. PSZ ceramic materials are employed in a wide range of biomedical and industrial products, including orthopedic joint implants, dental implants, battery manufacturing tooling, and oilfield equipment. The advantageous properties possessed by PSZ materials result from the specialized material microstructure which consists substantially of metastable, tetragonal domains of a critical size contained within cubic matrix grains. These PSZ materials are typically produced by combining zirconia with an oxide stabilizing agent such as MgO or CaO, usually by mixing monoclinic zirconia with the stabilizing oxide or a precursory material such as MgCO₃ or CaCO₃ to form a ceramic powder mixture. However, this mixing process does not induce the stabilization of the zirconia material required to attain the desired PSZ microstructure. Instead, the ceramic mixture cannot be converted to the PSZ state until it has been heated to high temperatures, typically during the process of sintering a formed ceramic body.

Prior methods for producing Mg-PSZ ceramics utilized raw materials consisting of monoclinic zirconia and an oxide stabilizing agent source such as MgO, MgCO₃, CaO, and CaCO₃. Monoclinic zirconia and the oxide stabilizing agent source are mixed and/or milled together to form a mixture of zirconia and stabilizing materials where the zirconia is not yet stabilized by the stabilizing agent. In cases where the oxide stabilizing agent source is an oxide precursor, such as MgCO₃ or CaCO₃, the mixture may be calcined to form mixed oxide powder of zirconia and oxide stabilizing agent where the zirconia remains unstabilized by the oxide stabilizing agent. Consequently, such a process does not yield PSZ ceramic powder (not pre-alloyed).

The non-stabilized oxide powder mixture is then processed to a final powder, used to form a ceramic body, and the ceramic body is heated to form a sintered PSZ ceramic material. Such a process, by necessity, involves heating of the non-stabilized oxide mixture to temperatures exceeding the cubic phase transformation temperature. To attain desired PSZ microstructure the heating process must allow tetragonal precipitates to form within cubic matrix grains, thus heat treatment conditions must be tailored to allow optimal formation and growth of tetragonal precipitates.

Sintered PSZ ceramic bodies can be used to manufacture components of significant strength and toughness for applications such as biomedical and industrial ceramics. Ceramic biomedical implants are known to have been produced via conventional PSZ processing methods, for example, they were produced using powder which is a simple mixture of MgO and ZrO₂. An example of such a process is described in U.S. Pat. No. 9,162,008. In such a process, MgO and ZrO₂ are mixed/milled together to produce non-stabilized powder mixture, powder is formed into ceramic body which may be machined in a green or bisqued state, then the formed ceramic body is sintered to the cubic transformation temperature range to yield PSZ sintered ceramic, and additional processing activities (machining, polishing, coating, etc.) are performed to produce final biomedical implant.

However, standard methods for producing PSZ ceramics have notable limitations. For example, disparities in mixing/milling behavior between zirconia and oxide stabilizing agent sources result in mill slurries which are difficult to process, including variable slurry viscosity and filtering issues. And, incomplete mixing of zirconia and oxide stabilizing agents can create issues during sintering. More specifically, differences in diffusion of zirconia and MgO/CaO can result in the formation of pores during sintering, affecting microstructure and limiting density, and porosity impacts surface finish and wear of PSZ ceramic components. Also, heat treatment cycles can be very energy intensive, require prolonged firing times, and as a result may adversely affect PSZ material properties if not completed correctly. For example, heat treatment is carried out at high temperatures (1700° C. to 1800° C.) with long cycle times, with the potential risk of overfiring above cubic phase transition temperature, which would adversely impact material strength.

The inability of the ceramic powder mixture to attain PSZ form and be processed as a stabilized zirconia powder creates certain limitations on the processing of these materials. Conversely, using a pre-alloyed PSZ powder facilitates easier production of sintered PSZ ceramic bodies and should impart improved properties to the sintered components.

Disclosed in embodiments herein are methods for producing toughened zirconia materials including: (a) processing zirconia and an oxide stabilizing agent materials to produce a partially stabilized zirconia (PSZ) powder, where the oxide stabilizer is dissolved into the zirconia microstructure, resulting in a pre-alloyed PSZ powder; and (b) using the pre-alloyed PSZ powder to produce a sintered PSZ body, made of the pre-alloyed PSZ powder, which remains stabilized and largely free of monoclinic content.

Further disclosed in embodiments herein is the use of the methods for the production of sintered PSZ ceramic bodies with improved properties such as such as increased strength and toughness.

Also disclosed herein is the use of the methods disclosed in the production of materials for biomedical (e.g., prostheses) and industrial components.

The various embodiments described herein are not intended to limit the disclosure to those embodiments described. On the contrary, the intent is to cover all alternatives, modifications, and equivalents as may be included within the spirit and scope of the various embodiments and equivalents set forth.

### DETAILED DESCRIPTION

The following acronyms and keywords are used in the description and claims:

Zirconia: Zirconium oxide (ZrO₂);

Magnesia: Magnesium oxide (MgO);

Calcia: Calcium oxide (CaO);

PSZ: Partially stabilized zirconia microstructure substantially consisting of metastable, tetragonal domains of a critical size contained within cubic matrix grains;

Mg-PSZ: Partially stabilized zirconia consisting of MgO dissolved in the zirconia structure as the stabilizing oxide agent;

Ca-PSZ: Partially stabilized zirconia consisting of CaO dissolved in the zirconia structure as the stabilizing oxide agent;

TZP: Tetragonal zirconia polycrystal microstructure; and

XRD: X-Ray Diffractometry characterization method for examining material crystal structure.

The basic operations for a method of producing toughened zirconia materials, possibly for prostheses, includes: (a) processing zirconia and oxide stabilizing agent materials to produce a partially stabilized zirconia (PSZ) powder, where the oxide stabilizer is dissolved into the zirconia microstructure, resulting in a pre-alloyed PSZ powder; and (b) processing the pre-alloyed PSZ powder to produce sintered PSZ bodies, with the pre-alloyed PSZ powder in the PSZ bodies remaining stabilized and largely free of monoclinic content throughout the process. For example, the level of monoclinic content in the sintered PSZ bodies would be <15%, and more preferably <2%. Subsequently, the sintered PSZ bodies may be employed to manufacture products for applications such as biomedical ceramics and industrial ceramics. Moreover, alternate and/or simplified traditional ceramics processing methods and techniques are facilitated by the pre-alloying of the PSZ material and producing sintered ceramic bodies with improved properties such as increased strength and toughness.

The process steps encompassed by this method may further include selecting raw materials from a zirconia source, such as monoclinic zirconia or zirconium chemicals, and an oxide stabilizing agent source, such as MgO or CaCO₃ and then mixing or otherwise combining the raw materials to prepare a mixture of desired composition. Next, the prepared mixture is treated in order to elicit the reaction or dissolution of the oxide stabilizer into the zirconia microstructure, resulting in a pre-alloyed PSZ material. The treatment applied to the prepared mixture to facilitate migration of the oxide stabilizer into the zirconia microstructure may be thermal or chemical.

Thermal treatments could include heating the zirconia-stabilizing oxide mixture to melt both constituents and induce integration of the stabilizing oxide into the zirconia microstructure. Thermal treatment is anticipated to be in the range of 2700° C. to 3200° C. for time periods of <1 second (thermal spray) up to 4 hours. Chemical treatments include chemical reaction of the zirconia-stabilizing oxide mixture to induce integration of stabilizing oxide into zirconia microstructure. Chemical treatments may be accomplished by reaction with gel-forming zirconium chemicals, such as zirconium propoxides.

Another aspect of the process includes maintaining conditions such that the PSZ powder retains a stabilized microstructure throughout any subsequent processing operation(s). For example, the thermal treatment products may be rapidly cooled to retain the heated microstructure as powder cools to ambient temperature. And, rapid cooling may also be employed during calcining above cubic/tetragonal phase range for chemical treatment products to prevent separation of the oxide stabilizers from the zirconia. Low temperature calcining (about 1200° C. to about 1500° C.) is used chemical treatment products to prevent separation of the oxide stabilizers from the zirconia. Alternatively, freeze drying at temperatures less than 0° C. and low temperature calcining may be employed for chemical treatment products to prevent separation of the oxide stabilizers from the zirconia.

Additional processing operations may be performed to transform PSZ powder into sintered ceramic bodies with the powder remaining substantially stabilized and free of monoclinic zirconia content. Use of the pre-alloyed PSZ powder affords the ability to use standard and simplified or alternate versions of traditional ceramic processing methods. The forming methods for which improved processing is achieved include slip casting, injection molding, and extrusion. Pre-forming processing techniques such as milling and filtering may also be improved and firing stages such as sintering could be improved as well. The anticipated improvements in pre-forming, forming and firing result from the pre-alloyed material being less susceptible to reaction with water or liquids. For example, the PSZ can be milled or other pulverized to reduce PSZ powder particle size and enhance forming behavior of the processed powder. The processed powder may then be formed to produce ceramic bodies and then sintering of formed bodies produced from PSZ powder.

And, as indicated previously, the pre-alloyed PSZ powders may be used for or as ceramic components in applications such as biomedical ceramics and structural ceramics using the sintered ceramic powders, including components that may possess improved properties. The improved properties, compared to conventional biomedical ceramics, are anticipated to include improved strength and toughness as a result of a more thorough and uniform dispersion of the oxide stabilizer into the zirconia microstructure.

Having generally described methods for the production of high-strength, high-toughness partially stabilized zirconia, the following description is directed to a more detailed disclosure of methods described above, including methods for the production of PSZ ceramic materials using zirconia and oxide stabilizing agents that are pre-alloyed to yield PSZ ceramic powder. As the disclosure above suggested, pre-alloying of zirconia and oxide stabilizing agent improves ease of processing and final mechanical properties of PSZ materials. Pre-alloying ensures comprehensive mixtures of zirconia and oxide stabilizing agent by limiting the presences of free oxide stabilizing agent in the mixture by ensuring incorporation into zirconia structure, and the microstructure will also be largely free of monoclinic content. Pre-alloying also facilitates easier milling and filtration of ceramic powder slurry during processing, where the PSZ powder behaves differently than non-stabilized comprised of zirconia plus an oxide stabilizing agent. In turn, pre-alloying also allows for use of alternate sintering and heat treatment conditions, as it removes the requirements and constraints associated with cubic phase transformation and formation of tetragonal precipitates. Thus the sintering and heat treating of pre-alloyed zirconia and oxide stabilizing agent enable the use of short duration, less energy intensive, and less expensive heating cycles. At the same time pre-alloying reduces risks of potentially overfiring materials and reducing strength of the fired materials. Accordingly, use of more favorable heating conditions facilitated by pre-alloying can result in improved strength and toughness of sintered PSZ materials.

Pre-Alloyed PSZ Material Production Methods

Production of pre-alloyed PSZ materials starts with raw material selection. One material selection step is the zirconia source, which may be monoclinic zirconia or zirconium precursor chemicals. another selection step is the oxide stabilizing agent source, which includes oxide materials (e.g., MgO, CaO) and precursor chemicals (e.g., Mg/Ca carbonate, nitrate, sulfate, acetate, and others). Additional raw material selection factors may include determining the raw material type by use of thermal or chemical treatments, assessing the purity of raw materials as may be established by requirements of the intended end product. For example, there may be raw material purity requirements for end products such as orthopedic implants, or chemical purity guidelines. And, there may be mechanical property requirements that dictate the use of particular raw materials and/or particular ratios of raw materials for the alloy.

Once the raw materials are selected, the oxide stabilizer and zirconia microstructure materials are mixed and processed to form the desired alloy ratio. As noted above, there are two methods to elicit the reaction or dissolution of the oxide stabilizer into the zirconia microstructure for the desired alloy; thermal treatment and chemical treatment. In the thermal treatment method, the mixture of zirconia and oxide stabilizing agent is heated to melt both constituents and induce integration of stabilizing oxide into the zirconia microstructure. The heated mixture is then cooled rapidly to retain the stabilized microstructure developed during heating process. Among the types of treatments that may be used are induction or electric arc fusion and thermal spray.

In the chemical treatment method, a chemical reaction is produced between zirconia and the oxide stabilizing agent to chemically induce integration of the stabilizing oxide into the zirconia microstructure. To accomplish the integration, the chemistry and temperature conditions are controlled to ensure retention of the stabilized zirconia microstructure. Heating processes may be used in treatment of the materials. However, as contracted to the thermal treatment described above, heating in the chemical method does not rely on complete melting of zirconia and oxide stabilizing agent to elicit reaction/dissolution of oxide stabilizing agent into zirconia microstructure. Chemical treatment types include gel synthesis, co-precipitation, freeze drying, thermal spray heating (with incomplete melting), microwave heating (with incomplete melting) and fast furnacing (with incomplete melting).

More specifically, for the thermal treatment process, the mixture of zirconia and oxide stabilizing agent source materials are prepared at the ratio necessary to yield desired PSZ product composition, for example:

Mg-PSZ: 2.5-wt % to 4.0-wt % MgO in ZrO₂; or

Ca-PSZ: 2.5-wt % to 7.0-wt % CaO in ZrO₂

Preparation of the mixtures may be accomplished by methods such as blending (e.g., ribbon blending, v-blending, cone blending, etc.) or milling (e.g., jar milling, vibratory milling, etc.). Some subsequent treatments of the mixtures (e.g., thermal spray) may require the mixture to be intimately mixed and extremely uniform, so chemical mixing may be preferred.

Once adequately mixed, the powder mixture is subjected to pre-alloying treatment. Using techniques such as induction, electric arc fusion or thermal spray, the powder mixture is heated above the melting points for both materials to produce a uniform mixture wherein the oxide stabilizing agent becomes reacted or dissolved in the zirconia microstructure. Reaction or dissolution of the oxide stabilizing agent in zirconia microstructure occurs above the cubic or cubic/tetragonal range, resulting in the formation of cubic or cubic/tetragonal microstructure PSZ materials. Once thermally treated, the mixture is subjected to rapid cooling to retain the desired high temperature crystal structure (cubic, cubic/tetragonal). Cooling methods may include use of air, water, and solution cooling, and it is to me noted that to retain the desired crystal structure, Mg-PSZ requires faster cooling than Ca-PSZ.

Alternatively, for the chemical treatment process, the mixture of zirconia and oxide stabilizing agent source materials are again prepared at the ratio necessary to yield desired PSZ product composition, for example:

Mg-PSZ: 2.5-wt % to 4.0-wt % MgO in ZrO₂; or

Ca-PSZ: 2.5-wt % to 7.0-wt % CaO in ZrO₂

Mixtures of materials for use in chemical treatments methods will typically need to be very thoroughly and uniformly intermixed. Techniques to assure intimate and uniform mixtures may include gel formation, co-precipitation and solution formation. The chemical treatment methods may utilize heating to enable the alloy stabilization process to occur, but such methods do not rely on melting of both the oxide stabilizing agent and zirconia in order to achieve stabilization.

Next, the powder mixture is subjected to a pre-alloying treatment. Chemical treatment types include gel synthesis, co-precipitation, freeze drying, thermal spray heating (with incomplete melting), microwave heating (with incomplete melting) and fast furnacing (with incomplete melting). The later chemical treatments further utilize rapid cooling during calcining cycles of chemical treatment products (gel synthesis, co-precipitation, freeze dried powders) which have been heated into the cubic or cubic/tetragonal phase range to ensure retention of high temperature (cubic or cubic/tetragonal) crystal structures upon cooling and prevent separation of oxide stabilizing agent from zirconia microstructure (separate into MgO/CaO+ZrO₂). Here again, Mg-PSZ typically requires faster cooling than Ca-PSZ. Utilizing low temperature calcining conditions for chemical treatment of powder products prevents separation of oxide stabilizing agent from zirconia microstructure, and may include freeze drying of solutions.

Next, the PSZ powders generated by chemical and thermal pre-alloying treatments are further processed to enable forming and sintering of powder materials. The pre-alloyed powder materials are milled to reduce particle size and increase surface area of the material, for example, using a ball mill, attrition mill or vibratory mill. It will be appreciated that additional pre-mill processes may be necessary if pre-alloying treatments result in materials that are too large to mill. The pre-mill processing may include crushing and sizing prior to milling to increase milling efficiency.

In this dry milling process, granulate or dry mill dried PSZ powder materials yield PSZ powder suitable for use in forming processes. Advantageously, the milled powder retains the desirable PSZ structure throughout milling process, and stabilizing oxide agents remain contained withing zirconia structure, without any free MgO or CaO and largely free of monoclinic content. The absence of the free MgO or CaO in these scenarios indicates that the pre-alloying of the zirconia with the oxide stabilizing agent has been achieved. For example, it is contemplated that there should be no substantial amount of free MgO or CaO, meaning less than 0.5% free MgO or CaO, preferably less than 0.3% free MgO or CaO, and more preferably less than 0.1% free MgO or CaO in the milled powder.

A PSZ powder slurry may be produce and for used in milling. The lack of free MgO or CaO, resulting from pre-alloying process, allows for the creation of more stable slurries, where standard dispersants such as ammonium polyacrylate (e.g., Darvan® 821A) can be employed. Moreover, the enhanced slurry stability enables better dispersion and increased solids loading.

Milling PSZ powder materials to desirable particle size distribution and/or specific surface area is conducted and then the slurry is discharged for drying. This results in increased slurry stability and dispersions facilitate easier and more efficient milling of PSZ powders because of the avoidance of adverse effects associated with free MgO or CaO in the slurry. Moreover, the PSZ slurry facilitates easier filtration and size separation of powder slurry. As will be appreciated the dry and wet milling operations may also be used in combination to achieve the desired pre-alloyed PSZ powder characteristics (e.g., particle size distribution and/or specific surface area).

Subsequently, the PSZ powder materials are employed in conventional ceramic forming processes to yield formed PSZ ceramic bodies, for example, pressing, extrusion and slip casting. Furthermore, the formed bodies can be additionally processed to alter their shapes and dimensions using techniques such as green machining and bisque machining. And again, the PSZ ceramic material retains PSZ structure throughout the forming process, where stabilizing oxide agents remain contained within zirconia structure, no free MgO or CaO is present and the material remains largely free of monoclinic content.

The pre-alloyed formed PSZ powder ceramic materials may then be sintered or heated to form PSZ ceramic components. Sintering results in a densification of the formed body, and yields sintered PSZ ceramic components with high strength and high toughness.

Also of interest are potential distinctions between sintering of traditional versus pre-alloyed PSZ powder materials. Traditional sintering of PSZ materials requires heating a non-stabilized oxide mixture to temperatures near or above the cubic transformation temperature. Formation of PSZ microstructure in the traditional materials requires that the sintering process be executed in manner that allows tetragonal precipitates to form within a cubic matrix. To achieve this, specific heat treatment process parameters must be followed to facilitate formation and growth of tetragonal precipitates. For example, as previously noted, heat treatment cycles to 1700° C. to 1800° C. for significant durations are necessary to achieve cubic phase transition. And variations in diffusion behavior between stabilizing oxide agents and zirconia may, nonetheless, result in the formation of pores during sintering, reducing density and negatively affecting mechanical properties of the sintered ceramic.

In comparison, the sintering process for pre-alloyed PSZ materials is not constrained by the same heating requirements. Use of pre-alloyed PSZ materials eliminates the necessity of heating to a cubic transformation temperature range, as PSZ phase composition has already be developed by the pre-alloying process disclosed above. Not having to heat to cubic transformation temperature range may allow for use of lower sintering temperatures, where the only consideration for selection of heating cycle is ensuring desired densification during the sintering process. And, pre-alloyed PSZ powder materials can still be sintered at standard cubic transition temperature range, but intimate mixing and pre-alloying of the materials allow for the use of shortened hold times at temperature. Reduced sintering temperatures and hold times can also reduce the degree to which grains are enlarged during the sintering process, resulting in sintered ceramic materials with small grain sizes, and smaller grain sizes generally correlate with improved mechanical properties such as increased strength and toughness in pre-alloyed PSZ sintered ceramics.

In contrast to conventional PSZ processing methods utilizing powder that is a simple mixture of MgO and ZrO₂, using the methods disclosed herein, biomedical implants are produced with pre-alloyed PSZ processing methods. Such methods utilize stabilization treatments, as described above, to produce the pre-alloyed PSZ ceramic powder. The pre-alloyed PSZ ceramic powder is then used to complete the remaining ceramic process steps to produce finished biomedical implants, with process steps benefitting from use of pre-alloyed PSZ powder. For example, slurry preparation, milling, and filtration processes benefit from increased ease of processing the pre-alloyed PSZ powder, which can result in improved properties for the final biomedical implant. Traditional forming methods can be readily used with pre-alloyed PSZ powder; the material performs well in isostatic pressing, while the lack of free oxide stabilizing agent improves processing conditions in forming applications such as slip casting and gel casting. Moreover, materials formed from pre-alloyed PSZ powders hold up well during green and bisque machining operations, and pre-alloyed PSZ formed bodies are capable of sintering to high densities with limited porosity using reduced sintering times and temperatures, resulting in energy savings. And, reduced grain sizes of sintered ceramics made from pre-alloyed PSZ powder can result in sintered PSZ materials with increased strength, increased toughness, and are capable of achieving better polishes with lower polished surface roughness. In dental ceramic implants, for example, lower sintering temperatures may result in better aesthetics for the final implant; options for bisque and green machining, grinding, and polishing make pre-alloyed PSZ an excellent candidate for use in dental labs.

While the methods and materials disclosed herein have biomedical applications, the production of ceramic material products using PSZ powder materials has general industrial applications as well. In such applications, the higher material densities, and improved mechanical properties, achievable using pre-alloyed PSZ make the materials suitable for wear component and tooling applications. Furthermore, the reduced sintering temperatures necessary to produce final PSZ materials may increase commercial accessibility of the pre-alloyed PSZ materials disclosed herein. Commercial production of PSZ materials can be constrained by high sintering temperatures required (much higher than TZP materials), and as a result of the pre-alloyed materials there are potential opportunities to replace TZP materials, such as yttria tetragonal zirconia polycrystal (Y-TZP) industrial components, with PSZ materials that possess higher toughness and are more easily machined in the sintered state. The increased utility of multiple forming techniques, such as slip casting and gel casting, enable such techniques to be employed in forming complex component shapes and configurations. And, the machinability of PSZ materials in green, bisque, and sintered states further facilitates production of complex shapes and configurations.

Having disclosed methods for the preparation of high-strength and high-toughness PSZ materials, particularly those by incorporating a ceramic powder in which the stabilizing oxide agent is pre-alloyed with the zirconia material powder to produce a pre-stabilized ceramic powder, the following examples are presented.

### EXAMPLES

The first example presented is directed to rapidly-cooled fused cast Mg-PSZ processing. In this example, the Mg-PSZ powder is prepared in accordance with the steps above, and the by pre-alloying treatment used is induction fusion and rapid cooling. Moreover, the cooling was accomplished with atomization and water cooling of the induction-fused material. Next, the fused Mg-PSZ powder was analyzed via x-ray diffraction (XRD) and determined to exhibit a primarily cubic+tetragonal (or non-monoclinic) structure, and exemplary data from an XRD analysis is found the following table, which characterizes pre-stabilized Mg-PSZ material and traditional Mg-PSZ material.

Magnesia-Zirconia Powder Phase Composition
via Semi-Quantitative X-Ray Diffractometry

| | | Traditional |
|---|---|---|
| | Pre-Stabilized | (Non-Stabilized) |
| Zirconia - Monoclinic (Baddeleyite) | 0.0% | 89.0% |
| Zirconia - Cubic | 88.1% | 0.0% |
| Zirconia - Tetragonal | 11.9% | 7.6% |
| MgO | 0.0% | 3.4% |

And, the pre-alloyed PSZ powder was easily dispersed for wet milling, milled to an average particle size below 1 micron, and filtered through a 10-micron filter. The milled powder could also be sized by sedimentation below 0.5 to 1.0 microns. X-ray diffraction characterization of the milled pre-alloyed PSZ powder showed that powder was still cubic in structure after milling as indicated in the table above.

The milled PSZ powder was used to prepare a stable slip cast slurry, and then a thick (approx. 1.25 cm thickness), high density part was cast from the slurry. The cast part was subsequently sintered using a short hold (15 minutes) at 1700° C., followed by a heat treatment stage (1340° C. for 2 hours). The density of the sintered part was measured as 5.845 g/cm³, or greater than 99.5% of its theoretical density. Further characterization of the sintered part using XRD revealed a phase composition mixture of cubic and tetragonal (target phase composition) structure.

In the second example, gel-prepared Mg-PSZ processing was employed where the Mg-PSZ powder was prepared by a gel synthesis process. The resulting gel material was calcined in the cubic/tetragonal phase temperature range and then rapidly cooled to form final pre-alloyed Mg-PSZ powder. Characterization was conducted using XRD on the calcined Mg-PSZ powder, and the XRD pattern indicated no free MgO was present, demonstrating that MgO was dissolved in the zirconia microstructure. The calcined chemically pre-alloyed Mg-PSZ powder behaved similarly to fused pre-alloyed Mg-PSZ powder in subsequent milling and processing operations.

It should be understood that various changes and modifications to the embodiments described herein will be apparent to those skilled in the art. Such changes and modifications can be made without departing from the spirit and scope of the present disclosure and without diminishing its intended advantages. It is therefore anticipated that all such changes and modifications be covered by the instant application.

What is claimed is:

1. A method for producing toughened zirconia materials including:
(a) processing zirconia and an oxide stabilizing agent materials to produce a partially stabilized zirconia (PSZ) powder, where the oxide stabilizing agent is dissolved into the zirconia microstructure, resulting in a pre-alloyed PSZ powder; and
(b) using the pre-alloyed PSZ powder to produce a sintered PSZ body, made of the pre-alloyed PSZ powder, which remains stabilized and largely free of monoclinic content.

2. The method according to claim 1, wherein the step of processing zirconia and an oxide stabilizing agent to produce a pre-alloyed PSZ powder includes:
mixing the zirconia and the oxide stabilizing agent to prepare a mixture of a desired composition; and
treating the prepared mixture to elicit the reaction or dissolution of the oxide stabilizing agent into the zirconia microstructure, resulting in a pre-alloyed PSZ material.

3. The method according to claim 2, wherein the step of treating the prepared mixture is a thermal or chemical based operation.

4. The method according to claim 2, wherein the step of treating the prepared mixture heats the mixture to melt both constituents and induce integration of the oxide stabilizing agent into the zirconia microstructure.

5. The method according to claim 2, wherein the step of treating the prepared mixture chemically reacts the zirconia-stabilizing oxide mixture to induce integration of the oxide stabilizing agent into zirconia microstructure.

6. The method according to claim 1 further including selecting raw materials from:
a zirconia source including monoclinic zirconia or zirconium chemicals; and
an oxide stabilizing agent source including MgO or CaCO₃.

7. The method according to claim 1, wherein the step of using the pre-alloyed PSZ powder further includes producing a ceramic body prosthetic implant or prosthetic implant component.

8. The method according to claim 7, wherein the oxide stabilizing agent is MgO and is reacted or dissolved in zirconia microstructure, with no substantial amount of free MgO remaining.

9. The method according to claim 8, wherein the sintered PSZ body comprises a ceramic body prosthetic implant or prosthetic implant component of Mg-PSZ ceramic.

10. The method according to claim 8, wherein the sintered PSZ body is formed using a method selected from the group consisting of: cold isostatic pressing, slip casting, and pressure casting.

11. The method according to claim 10, wherein an initial green body made from the pre-alloyed PSZ powder is machined to near final shape, or the body is bisque fired and then machined, with the machined body then being fired using a PSZ sintering cycle.

12. The method according to claim 1, wherein the sintered PSZ body is prepared from a PSZ powder and where the powder is pre-stabilized PSZ using CaO as the oxide stabilizing agent, which is reacted or dissolved in zirconia microstructure with no substantial amount of free CaO.

13. A biomedical implant made from pre-alloyed PSZ powder produced in accordance with the method of claim 1.

14. A partially stabilized zirconia material produced in accordance with the following method:
(a) processing zirconia and an oxide stabilizing agent materials to produce a partially stabilized zirconia (PSZ) powder, where the oxide stabilizing agent is dissolved into the zirconia microstructure, resulting in a pre-alloyed PSZ powder, and wherein the step of processing zirconia and an oxide stabilizing agent to produce a pre-alloyed PSZ powder includes:
1) mixing the zirconia and the oxide stabilizing agent to prepare a mixture of a desired composition; and
2) treating the prepared mixture to elicit the reaction or dissolution of the oxide stabilizing agent into the zirconia microstructure, resulting in a pre-alloyed PSZ material;
(b) using the pre-alloyed PSZ powder, producing a sintered PSZ body, made of the pre-alloyed PSZ powder, which remains stabilized and largely free of monoclinic content, including producing a ceramic body prosthetic implant or prosthetic implant component.

15. The material produced in accordance with the method of claim 14, wherein the sintered PSZ body is prepared from a PSZ powder and where the powder is pre-stabilized PSZ using CaO as the oxide stabilizing agent, which is reacted or dissolved in zirconia microstructure with no substantial amount of free CaO.
